(19) United States
(12) Patent Application Publication
(10) Pub. No.: US 2025/0135161 A1
(43) Pub. Date: May 1, 2025

(54) GRIPPER FOR AN ENDOVASCULAR SYSTEM

(71) Applicant: UAB Inovatyvi Medicina, Kaunas (LT)

(72) Inventors: Giedrius KEREŽIS, Kaunas (LT); Remigijus GRIŠIUS, Kaunas (LT); Vaidas LABUNSKAS, Kaunas (LT); Vilius DAMBRAUSKAS, Kaunas (LT)

(73) Assignee: UAB Inovatyvi Medicina, Kaunas (LT)

(21) Appl. No.: 18/837,147

(22) PCT Filed: Mar. 7, 2023
(86) PCT No.: PCT/EP2023/055716

§ 371 Date: Aug. 8, 2024

(30) Foreign Application Priority Data
Apr. 1, 2022 (DE) 102022107835.3

(51) Int. Cl. A61M 25/09

(52) U.S. Cl. CPC A61M 25/09; A61M 2025/09116

(57) ABSTRACT

A gripper (100) for gripping an elongated member (6) of an endovascular system (300), wherein the gripper (100) comprises: a first gripping component (2) configured to contact at least a first portion of a said elongated member (6) on a first side of said elongated member (6): a second gripping component (3) configured to contact at least the first portion of said elongated member (6) on a second side of said elongated member (6), wherein the first side is different from the second side: a guide (1) configured to guide, during a movement of the second gripping component (3), at least a first portion of the second gripping component (3); and an actuating component (5) coupled to the second gripping component (3), wherein the second gripping component (3) is moveable between a first position and a second position based on an actuating force provided to the second gripping component (3) via the actuating component (5), and wherein at least the first portion of the second gripping component (3) is guideable by the guide (1) during movement of the second gripping component (3) between the first position and the second position; wherein the first gripping component (2) is configured to stay stationary or substantially stationary with respect to the guide (1), wherein the second gripping component (3) comprises a first surface opposite to a first surface of the first gripping component (2), wherein, when the second gripping component (3) is in the first position, said elongated member (6) is grippable between the first surface of the first gripping component (2) and the first surface of the second gripping component (3), and wherein, when the second gripping component (3) is in the second position, said elongated member (6) is not grippable between the first surface of the first gripping component (2) and the first surface of the second gripping component (3).

### FIELD OF THE INVENTION

The present invention generally relates to a gripper for gripping an elongated member of an endovascular system comprising a first and a second gripping component, a guide and an actuating component, wherein said elongated member is grippable by the first and second gripping components.

### BACKGROUND TO THE INVENTION

Endovascular specialists (for example (endo-) vascular surgeons, (interventional) cardiologists, (interventional) radiologists etc.) train, practice and develop intuitive skills to handle surgical tools. One of these tools is a gripper which allows for an elongated member, such as a catheter or a guide wire, to stay in a relatively fixed position while the specialist uses the elongated member.

Currently, existing grippers are suitable for only a very selected number of elongated members from the range that endovascular specialists use. For example, grippers may only be able to accommodate catheters or only be able to accommodate guide wires. Furthermore, known grippers, such as the ones disclosed in US 2007/0219467 A1, use shear stress to grip the elongated member in place. This can lead to excess stress loads and forces being placed upon the elongated member, thereby reducing the lifetime of the elongated member, and possibly leading to a breaking of the elongated member during use on a patient.

There is therefore a need for improvements of grippers within endovascular systems.

### SUMMARY OF THE INVENTION

The invention is set out in the independent claims. Preferred embodiments of the invention are set out in the dependent claims.

According to a first aspect, we describe a gripper for gripping an elongated member of an endovascular system. The gripper comprises a first gripping component configured to contact at least a first portion of a said elongated member on a first side of said elongated member, a second gripping component configured to contact at least the first portion of said elongated member on a second side of said elongated member, wherein the first side is different from the second side, a guide configured to guide, during a movement of the second gripping component, at least a first portion of the second gripping component and an actuating component coupled to the second gripping component, wherein the second gripping component is moveable between a first position and a second position based on an actuating force provided to the second gripping component via the actuating component, and wherein at least the first portion of the second gripping component is guidable by the guide during movement of the second gripping component between the first position and the second position. The first gripping component is configured to stay stationary or substantially stationary with respect to the guide. The second gripping component comprises a first surface opposite to a first surface of the first gripping component. When the second gripping component is in the first position, said elongated member is grippable between the first surface of the first gripping component and the first surface of the second gripping component. When the second gripping component is in the second position, said elongated member is not grippable between the first surface of the first gripping component and the first surface of the second gripping component.

The first gripping component is configured to contact at least a first portion of the elongated member on a first side of said elongated member and the second gripping component is configured to contact a second side of said elongated member, wherein the first side is different from the second side. This results in the elongated member being contacted by the gripping components on two different sides, thereby allowing for the elongated member to be gripped by the two gripping components. The gripping components may be any suitable shape which allows for said gripping components to contact at least the first portion of the elongated member such as, for example, cuboidal, prismoidal or a bespoke design. The gripping components may comprise any suitable material, as will be described in more detail below.

The guide may be of any suitable design which allows for the movement of the second gripping component to be guided during movement of said second gripping component. The guide may be U-shaped, L-shaped, two substantially vertical pieces which allow for the second gripping component to be guided, or any other bespoke shape.

The actuating component coupled to the second gripping component allows for the second gripping component to be moved between a first and a second position via an actuating force. The actuating force may be a mechanical force, a resilient force, a gravitational force, a magnetic force or any other type of force, or a combination thereof.

The first gripping component is configured to stay stationary or substantially stationary with respect to the guide. This may allow for the first gripping component to be moved at the same time as the guide should the guide be moved and vice versa. The first gripping component may be directly or indirectly coupled to the guide via any suitable coupling means.

The second gripping component is, as described above, moveable between a first and a second position via an actuating force provided to the second gripping component via the actuating component. When the second gripping component is in the first position, the elongated member is grippable between a first surface of the first gripping component and a first surface of the second gripping component. This allows for the elongated member to be gripped in place should an endovascular specialist, or any person, be using said gripper. This allows for the elongated member to be secured in place. In the second position, the elongated member is not grippable between a first surface of the first gripping component and a first surface of the second gripping component. This allows for the elongated member to be moved within the gripper, for an elongated member to be removed from the gripper entirely or for an elongated member to be inserted into the gripper.

Furthermore, as the elongated member is grippable between a first surface of the first gripping component and a first surface of the second gripping component, this may mean that the elongated member is grippable via a compressive force. This may allow for a more secure gripping and/or securing of the elongated member and may allow for the lifetime of the elongated member to be increased, as it is not subjected to shear or tension forces.

In some examples, the first surface of the first gripping component comprises a first recess configured to accommodate at least the first portion of said elongated member on the first side of said elongated member. The recess may be of any suitable design that allows for at least the first portion of the elongated member to be accommodated. The recess may be U-shaped, semi cylindrical, prismoidal, have varying dimensions or be of any bespoke design. In some examples, the recess comprises a plurality of shapes at different locations, which may correspond to differing sections of the elongated member. This may allow for the first gripping component to be designed in such a way that allows for said first gripping component to be able to accommodate a plurality of different types of elongated member and/or if the elongated member has different cross-sections at different sections. This may improve the versatility of the gripper.

In some examples, the first recess is V-shaped. The use of a V-shape may mean that a single first gripping component can be used for a plurality of different types of elongated member as the V-shape allows, e.g., for elongated members of different dimensions to be accommodated by the first recess. The use of a V-shape may also allow for a plurality of contact points around the circumference of the elongated member, thereby decreasing the likelihood of the elongated member slipping during use of the gripper.

In some examples, the first surface of the second gripping component comprises a second recess configured to accommodate at least the first portion of said elongated member on the second side of said elongated member. The recess may be of any suitable design that allows for at least the first portion of the elongated member to be accommodated. The recess may be U-shaped, semi cylindrical, prismoidal, have varying dimensions or be of any bespoke design. In some examples, the recess comprises a plurality of shapes which may correspond to differing sections of the elongated member. This may allow for the second gripping component to be designed in such a way that allows for said second gripping component to be able to accommodate a plurality of different types of elongated member and/or if the elongated member has different cross-sections at different sections. This may improve the versatility of the gripper.

In some examples, the second recess is V-shaped. This may allow for a plurality of contact points around the circumference of the elongated member, thereby decreasing the likelihood of the elongated member slipping during use of the gripper. In some examples, at least the first portion of said elongated member is contactable, via the first surface of the first gripping component on the first side of said elongated member and the first surface of the second gripping component on the second side of said elongated member, at four contact lines via the first and second recesses. The use of contact lines may allow for the compressive force to be exerted along a portion of the elongated member, thereby reducing the force that is exerted on any single portion. This may allow for the elongated member to have a longer lifespan, as the force is not focused at a single point. It may also allow for a more secure gripping of the elongated member as the elongated member is contacted along a portion of said elongated member by the first and second gripping components, thereby increasing the area of contact by the gripping components.

In some examples, the four contact lines are each separated by 90° (or approximately) 90°, and wherein the four contact lines are parallel to one another. This may allow for the compressive forces to be shared equally throughout the elongated member, thereby increasing the lifespan of the elongated member. Furthermore, as the contact lines are equally spaced, this may allow for a more secure gripping of the elongated member as the contact lines are not focused in a single area of the elongated member but shared equally around said elongated member.

In some examples, at least the first portion of said elongated member is contactable, via the first surface of the first gripping component on the first side of said elongated member and the first surface of the second gripping component on the second side of said elongated member, at four contact points via the first and second recesses. The use of contact points may allow for a gripper which requires a lower actuating force to be provided by the actuating component on the second gripping component in order to grip said elongated member as the gripper does not need to ensure that the elongated member is gripped along a contact line. As a lower force is exerted on the elongated member, this may increase the lifespan of the elongated member while still maintaining a suitable level of gripping by the gripper.

In some examples, the four contact points are each separated by 90° (or approximately) 90°, and wherein the four contact points span a quadrilateral around a central longitudinal axis of said elongated member. This may allow for the compressive forces to be shared equally throughout the elongated member, thereby increasing the lifespan of the elongated member. Furthermore, as the contact points are equally spaced, this may allow for a more secure gripping of the elongated member as the contact points are not focused in a single area of the elongated member but shared equally around said elongated member.

In some examples, the first surface of the second gripping component comprises a protrusion configured to contact at least the first portion of said elongated member on the second side of said elongated member. The protrusion may be of any suitable design that allows for at least the first portion of the elongated member to be contacted. The protrusion may be cuboidal, semi cylindrical, prismoidal, have varying dimensions or be of any bespoke design. In some examples, the protrusion comprises a plurality of shapes which may correspond to differing sections of the elongated member. This may allow for the second gripping component to be designed in such a way that allows for said second gripping component to be able to contact a plurality of different types of elongated member and/or if the elongated member has different cross-sections at different sections.

In some examples, the protrusion comprises a truncated V-shape. This may allow for the protrusion to comprise at least one relatively flat surface. The elongated member preferably contacts this relatively flat surface, thereby reducing possible unwanted forces to be exerted on the elongated member, that may otherwise be exerted by a non-flat surface, and therefore extend the lifespan of the elongated member. The use of the relatively flat surface may also allow for the protrusion to be able to contact a plurality of different cross-sections of elongated members, thereby increasing the versatility of the gripper.

In some examples, at least the first portion of said elongated member is contactable, via the first surface of the first gripping component on the first side of said elongated member and the first surface of the second gripping component on the second side of said elongated member, at two contact lines via the first recess and one contact line via the protrusion. The use of contact lines may allow for the compressive force to be exerted along a portion of the elongated member, thereby reducing the force that is exerted on any single portion. This may allow for the elongated member to have a longer lifespan as the force is not focused at a single point. It may also allow for a more secure gripping of the elongated member as the elongated member is contacted along a portion of said elongated member by the first and second gripping components, thereby increasing the area of contact by the gripping components.

In some examples, the three contact lines are each separated by 120° (or approximately) 120°, and wherein the three contact lines are parallel to one another. This may allow for the compressive forces to be shared equally throughout the elongated member, thereby increasing the lifespan of the elongated member. Furthermore, as the contact lines are equally spaced, this may allow for a more secure gripping of the elongated member as the contact lines are not focused in a single area of the elongated member but shared equally around said elongated member.

In some examples, at least the first portion of said elongated member is contactable, via the first surface of the first gripping component on the first side of said elongated member and the first surface of the second gripping component on the second side of said elongated member, at two contact points via the first recess and one contact point via the protrusion. The use of contact points may allow for a gripper which requires a lower actuating force to be provided by the actuating component on the second gripping component in order to grip said elongated member as the gripper does not need to ensure that the elongated member is gripped along a contact line. As a lower force is exerted on the elongated member, this may increase the lifespan of the elongated member while still maintaining a suitable level of gripping by the gripper.

In some examples, the three contact points are each separated by 120° (or approximately) 120°, and wherein the three contact points span a triangle around a central longitudinal axis of said elongated member. This may allow for the compressive forces to be shared equally throughout the elongated member, thereby increasing the lifespan of the elongated member. Furthermore, as the contact points are equally spaced, this may allow for a more secure gripping of the elongated member as the contact points are not focused in a single area of the elongated member but shared equally around said elongated member.

In some examples, the actuating component comprises a resilient member, in particular a spring. In some examples, the resilient member exerts a biasing force on the second gripping component, wherein the biasing force biases the second gripping component towards the first position i.e. the gripping position. This may allow for the elongated member to be gripped by the gripper without a user of the gripper needing to constantly keep the second gripping component in the first position. The resilient member may be any suitable resilient member such as, for example, a spring, a rubber band or any other suitable component that provides a resilient force.

In some examples, the guide comprises a through hole configured to receive said elongated member between the first surface of the first gripping component and the first surface of the second gripping component. This may allow for the elongated member to be accommodated by the guide, thereby providing an extra securing method as the elongated member movement of the elongated member may be restricted even when the second gripping component is in the second position i.e. the non-gripping position. It may also allow for a portion of the elongated member to extend beyond the guide and indeed, the gripper. More than one section of the guide may comprise a through hole, thereby allowing for the elongated member to extend beyond the guide, and the gripper, on at least two sides of the guide.

In some examples, at least the first portion of said elongated member is grippable between the first surface of the first gripping component and the first surface of the second gripping component. This may allow for a secure gripping of the elongated member, thereby reducing unwanted movement of the elongated member when the second gripping component is in the first position.

In some examples, the second gripping component and/or the guide comprises a photopolymer and/or comprises a 3D-printed portion. The use of a photopolymer and/or 3D printing allows for versatile components to be designed, i.e. components which are bespoke to the gripper, while allowing for said component to quickly set after production of said component. This may be particularly useful if a component breaks, so that it can quickly and easily be replaced via the 3D printing and/or the use of a photopolymer. Additionally or alternatively, at least a portion of the second gripping component and/or the guide may be constructed via plastic and/or metallic additive manufacturing and/or injection molding and/or electrical discharge machining, EDM, and/or any other suitable method. In some examples, the at least a portion of the second gripping component and/or the guide may comprise an element formed by at least one of these methods. Each of these methods may also be particularly useful if a component breaks, as the broken component can be quickly and easily replaced. In some examples, other parts of the gripper are at least partially formed and/or are at least partially constructed by and/or comprise an element constructed by at least one of the above-mentioned methods.

In some examples, the first gripping component comprises a metallic portion, wherein at least a portion of the metallic portion is configured to contact at least the first portion of said elongated member. The use of a metallic portion may allow for the elongated member to be secured in place during operation of the gripper, particularly if the metallic portion comprises a metallic material with a high coefficient of static friction. In some examples, the metallic portion at least partially comprises aluminum. In some examples, the portion of the elongated member that the metallic portion contacts comprises stainless steel and/or a nickel-titanium alloy (Nitinol). Furthermore, the at least first portion of the elongated member may comprise a coating which comprises a hydrophilic or hydrophobic polymer, dependent on the use of the elongated member and/or gripper. In some examples, an end of the elongated member may comprise a heavy metal such as, for example, gold or platinum for better visibility of said elongated member end.

In some examples, upon said movement of the second gripping component, based on the actuating force provided to the second gripping component via the actuating component, the first surface of the second gripping component is moveable towards the first surface of the first gripping component. This may allow for the elongated member to undergo a compressive force when gripped between the first and second gripping components. This may allow for a more secure gripping and/or securing of the elongated member and may allow for the lifetime of the elongated member to be increased as it is not subjected to shear or tension forces.

In some examples, the gripper further comprises an assembly pin configured to couple (i) the first gripping component and the guide to each other, and/or (ii) the first gripping component and a component external to the gripper to each other, and/or (iii) the guide and the component external to the gripper to each other. The assembly pin may be a pin, a nut and bolt, a screw, a hinge, a bayonet coupling, a welding or any other suitable type of coupling or any combination thereof. The coupling between the first gripping component and the guide may allow for the first gripping component to be directly coupled to the guide, thereby ensuring that the first gripping component is stationary or substantially stationary with respect to the guide. The coupling between the first gripping component and a component external to the gripper and/or the guide and the component external to the gripper may allow for the gripper to be secured to an external component. The external component may be, for example, a sensor, another part of the endovascular system, a securing component or any other type of component suitable in an endovascular system. In some examples, the external component may be a light visible to a user of the gripper, an indication on a screen visible to the user of the gripper, or some other visual method of showing that the gripper is in the “gripping” position. Additionally or alternatively, an audio and/or haptic feedback can be provided to the user via the external component. The assembly pin may additionally or alternatively restrict movement of the second gripping component when the second gripping component is in the second position. This may prevent the second gripping component from moving when the elongated member is not being gripped by the first and second gripping members.

In some examples, the guide comprises a first portion and a second portion, wherein the first portion is configured to guide at least the first portion of the second gripping component and to guide at least the first portion of said elongated member, wherein the second portion of the guide comprises a section configured to guide at least a second portion of said elongated member, and wherein the first and second portions of said elongated member are distinct portions. The first portion of the guide may be similar to the guide as described above. The second portion of the guide may allow for the second portion of the elongated member to be guided, thereby reducing unwanted movement of the elongated member when the second gripping component is in the first and/or second position. The second portion of the guide may guide a section of the elongated member that is not contactable by the first gripping component and/or the second gripping component.

In some examples, the gripper is situated in a disposable cassette. This may allow for the gripper to be easily inserted and/or extracted from the endovascular system during swap over of grippers. The use of a disposable cassette may also allow for an increase in hygiene as the disposable cassette may come into contact with body fluids and therefore need to be disposed after use. In some examples, the disposable cassette is sterilized before it is inserted into the endovascular system.

In some examples, the first gripping component and/or the second gripping component and/or the guide are at least partially coated in a polymer. The use of a polymer may allow for friction to be reduced between moving components, thereby improving the lifetime of the gripper due to a reduction in friction forces.

In some examples, the actuating force provided to the second gripping component via the actuating component is dependent on at least one parameter of said elongated member. The at least one parameter may be, for example, a material of the elongated member, a dimension of the elongated member, a usage of the elongated member, a resilience of the elongated member or any other suitable parameter, or, in case more parameters are taken into account, any combination thereof. The actuating force may be limited by the biasing force applied by the resilient member to the second gripping component. In some examples, if the elongated member is a catheter, the biasing force does not exceed 15 Newtons and if the elongated member is a guide wire, the biasing force does not exceed 5 Newtons.

In some examples, said elongated member is a guide wire and/or a catheter. This may be particularly advantageous in endovascular systems.

In some examples, the gripper further comprises a sensor configured to indicate to the external component an actuation of the actuating component. This sensor may be a position detector and detect if the second gripping component is in the first position or not in the first position. Resultantly, this may indicate to a user of the gripper that the second gripping component is in the first position. In some examples, the sensor is coupled to an indicator which may give a visual and/or audio and/or haptic indication to the user that the second gripping component is in the first position.

In some examples, the first gripping component is at least partially housed by the second gripping component. This may allow for a limitation of the travel of the second gripping component. This in turn may elongate the lifespan of the gripper as the components of the gripper may not be overstressed or undergo unwanted stresses or movements.

In some examples, the first and second gripping components are at least partially located within an inner guide, wherein the inner guide is located within the guide, and wherein the inner guide is moveable with respect to the guide. The inner guide, when moving with respect to the guide, may act as a form of suspension and cushioning for the elongated member. This may in turn reduce the stress and shear forces experienced by the elongated member during use of said member, thereby elongating the lifetime of the elongated member.

In some examples, the first and second gripping components are offset from each other along a longitudinal axis of the elongated member, and when the second gripping component moves between the first position and the second position, the second gripping component does not contact the first gripping component. This may allow for the elongated member to be gripped in a particularly strong manner as it needs to snake its way through the gripper.

In some examples, the first and second gripping components are arranged in a zipper configuration. This may allow for the elongated member to be gripped in a particularly strong manner as it needs to snake its way through the gripper.

In some examples, the guide is further configured to guide at least a portion of the actuating component and/or at least a first portion of the first gripping component. This may in turn elongate the lifespan of the gripper as the components are prevented from undergoing unwanted movements and therefore, unwanted or excess stresses and forces.

In some examples, the second gripping component is slideable past the first gripping component and the guide during the movement of the second gripping component between the first position and the second position and/or between the second position and the first position. This may allow for the second gripping component to comprise a portion placed between the first gripping component and the guide, and for this portion to be slideable past both the first gripping component and the guide. This may allow for unwanted movements of the second gripping component to be reduced when the actuating force is provided by the actuating component to the second gripping component. This in turn may increase the lifespan of the second gripping component and the gripper.

In some examples, the movement of the second gripping component towards the second position is limitable upon a contact between the first gripping component and the second gripping component. This may prevent the second gripping component and/or the first gripping component from undergoing excess forces which may reduce the lifespan of the gripping components. This may also limit the force exerted to the elongated member by the gripping components when the second gripping component is in the first position.

In some examples, the movement of the second gripping component towards the first position is limitable upon a contact between the second gripping component and said elongated member. In some examples, the movement is limitable once a predetermined force has been exerted onto the elongated member due to the movement of the second gripping component towards the first gripping component. This may allow for a longer lifespan of the second gripping component and/or the elongated member due to a reduction in unwanted forces being exerted on said second gripping component and/or elongated member. The predetermined force may be equivalent to the biasing force exerted by the resilient member on the second gripping component.

In some examples, a movement of said elongated member is limitable by the first recess and/or the second recess when the second gripping component is in the second position. This may allow for the elongated member to be moveable within the gripper while still maintaining control of the movement of the elongated member. This may be advantageous in scenarios where the elongated member is not needed to be moved a large amount and/or for readjustment of the position of the elongated member within the gripper and/or during a usage of the elongated member.

In some examples, the metallic portion comprises 7075-t6 aluminum. This may allow for the gripper to be lightweight while still maintaining strength and gripping performance.

In some examples, at least a portion of the first and/or second gripping component contactable with the elongated member comprises a coating layer. The coating layer may comprise one or more of: a rubber, a textile, a fabric, a polymer layer, a nonpolymer layer, a paint layer, a composite, and any other suitable material. This coating layer may provide an improved gripping (improved gripping resistance) of the elongated member while it is being gripped and help protect the elongated member, thereby extending the lifetime of said member. The coating layer may be glued, cured, or painted onto the portion of the first and/or second gripping component contactable with the elongated member comprising the coating layer, or may be a loose layer placed onto the portion of the first and/or second gripping component contactable with the elongated member. The coating layer may also minimize or avoid the risk of damaging the elongated member.

According to a second aspect, we describe an endovascular system comprising a first endovascular instrument, and a second endovascular instrument, wherein the first and/or second endovascular instrument comprises a gripper according to any one or more of the example implementations as described herein. The first and second endovascular instruments may be located in discrete locations and a movement of the first endovascular instrument may be translated to a corresponding movement in the second endovascular instrument.

In some examples, the endovascular system further comprises an elongated member, wherein the elongated member is a guide wire and/or a catheter. In some examples, the guide wire and/or catheter comprises stainless steel and/or a nickel-titanium alloy (Nitinol). Furthermore, the guide wire and/or catheter may comprise a coating which comprises a hydrophilic or hydrophobic polymer, dependent on the use of the guide wire and/or catheter and/or gripper. In some examples, an end of the guide wire and/or catheter may comprise a heavy metal such as, for example, gold or platinum for better visibility of said guide wire and/or catheter end.

In some examples, the first endovascular instrument is a first robotic endovascular instrument and/or the second endovascular instrument is a second robotic endovascular instrument.

Any advantages and features described in relation to the any of the above aspects and examples may be realized in any of the other aspects and examples described above.

It is clear to a person skilled in the art that certain features of the system set forth herein may be implemented under use of hardware (circuits), software means, or a combination thereof. The software means can be related to programmed microprocessors or a general computer, an ASIC (Application Specific Integrated Circuit) and/or DSPs (Digital Signal Processors). For example, a processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

Even if some of the aspects described above have been described in reference to the gripper and/or the endovascular system, these aspects may also apply to a method (in particular of gripping an elongated member with the gripper according to any one or more of the example implementations as described herein) and vice versa.

### BRIEF DESCRIPTION OF THE DRAWINGS

These and other aspects of the invention will now be further described, by way of example only, with reference to the accompanying figures, wherein like reference numerals refer to like parts, and in which:

FIG. 1 shows a perspective view of a schematic illustration of the gripper according to some example implementations as described herein;

FIG. 2 shows a cross-sectional view of a schematic illustration of parts of the gripper according to some example implementations as described herein;

FIG. 3 shows a cut-away view of a schematic illustration of the gripper according to some example implementations as described herein;

FIG. 4 shows a cross-sectional view of a schematic illustration of parts of the gripper according to some example implementations as described herein;

FIG. 5 shows a cross-sectional view of a schematic illustration of parts of the gripper according to some example implementations as described herein;

FIG. 6 shows cross-sectional views of a schematic illustration of parts of the gripper according to some example implementations as described herein;

FIG. 7 shows a cut-away view of a schematic illustration of the gripper according to some example implementations as described herein; and

FIG. 8 shows a schematic block diagram of an endovascular system according to some example implementations as described herein.

### DETAILED DESCRIPTION OF THE PREFERRED EMBODIMENTS

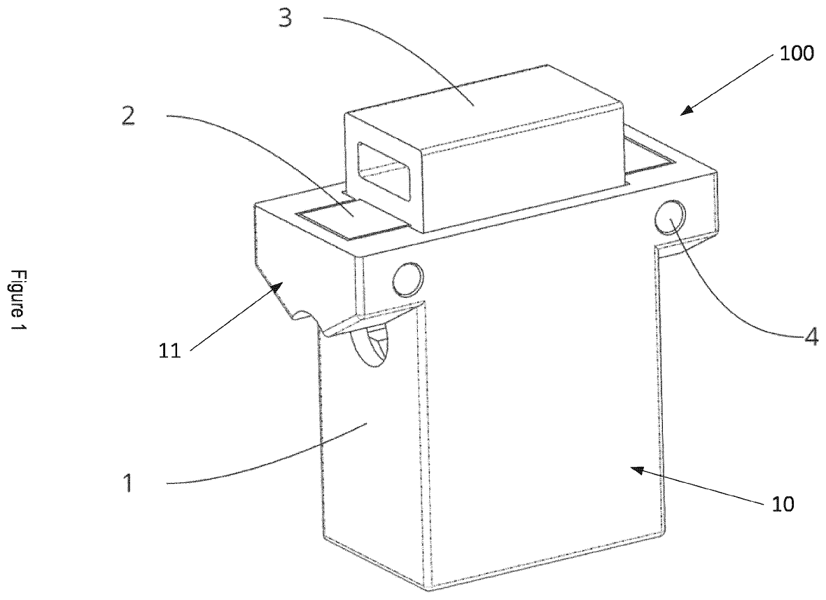

FIG. 1 shows a perspective view of a schematic illustration of the gripper according to some example implementations as described herein.

The gripper 100 comprises a guide 1, a first gripping component 2, a second gripping component 3 and a plurality of assembly pins 4.

The guide 1 is configured to guide at least the second gripping component 3, as will be described in more detail below. In this example, there are a plurality of assembly pins 4, but there may be a single assembly pin 4 or no assembly pins 4. The number of assembly pins 4 may vary depending on the design of the gripper 100. In some examples, the assembly pins 4 are configured to couple the first gripping component 2 to the guide 1, thereby ensuring that the first gripping component is stationary with respect to the guide 1. In some examples, the assembly pins 4 are configured to couple the first gripping component 2 and/or the guide 1 to an external component (not shown). The external component may be a sensor, a housing, an actuator, a part of an endovascular system or any other suitable component.

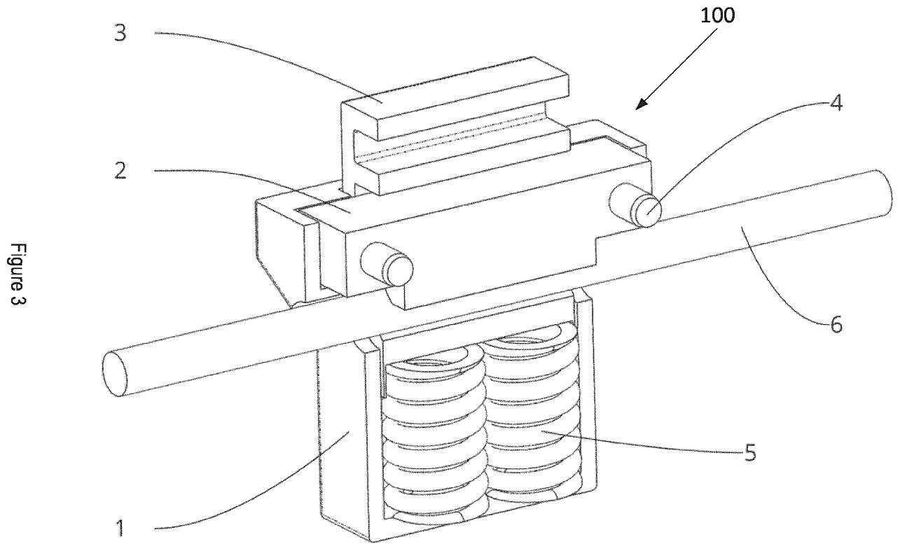

The guide 1 further comprises a first portion 10 and a second portion 11. The first portion 10 is configured to guide at least a portion of the second gripping component 2. The guide 1 may also guide an elongated member (see FIG. 3). The second portion 11 of the guide 1 is discrete from the first portion 10 and extends outwardly from the first portion 10 of the guide 1. The second portion 11 may also be configured to guide at least a portion of the elongated member, wherein the first 10 and second 11 portions of the guide 1 guide different portions of the elongated member. The first 10 and second 11 portions of the guide 1 are not limited to the design shown in FIG. 1 but may be of any suitable design which allows for the guiding of the elongated member and, in the case of the first portion, the guiding of at least the second griping component 3.

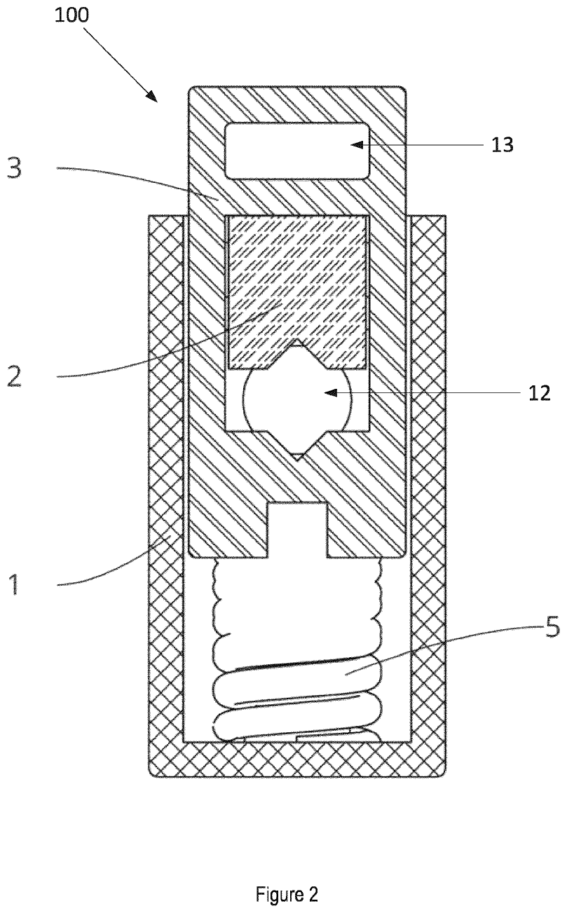

FIG. 2 shows a cross-sectional view of a schematic illustration of parts of the gripper according to some example implementations as described herein.

In FIG. 2, it can be seen that the second gripping component 3 is coupled to a resilient member 5, in this case a spring. The resilient member 5 may alternatively be any suitable component which provides a resilient force. The guide further comprises a through hole 12 configured to accommodate the elongated member and allow for the elongated member to travel through the gripper 100. The second gripping component 3 also comprises an opening 13 which extends beyond the guide 1. The opening 13 may allow for a weight reduction of the gripper 100. This may, in turn, result in an increase in performance as high inertia may reduce the performance of the gripper 100.

Furthermore, in this example, the guide 1 is configured to surround the spring 5, the first gripping component 2 and at least a portion of the second gripping component 3. This may allow for parts of the gripper 100 to be guided in a manner which reduces unwanted stress and shear forces during operation of the gripper 100, thereby extending the lifetime of the gripper 100. In some examples, the guide 1 guides only the second gripping component 3 and/or the spring 5.

The spring 5 coupled to the second gripping component allows for the second gripping component to be moveable with respect to the guide 1. The resilient force provided by the spring 5 preferably biases a first surface of the second gripping component 3 towards a first surface of the first gripping component 2, wherein the biasing results in the elongated member being gripped between the first 2 and second 3 gripping components, as will be described in more detail below. In some examples, a force provided by a pneumatic cylinder and/or an electric motor and/or any other suitable component against the resilient force provided by the spring 5 allows for the first surface of the second gripping component 3 and the first surface of the first gripping component 2 to be moved away from each other, thereby allowing for the elongated member to be ungripped (released) and allow for said elongated member to be moved within the gripper 100 or removed from the gripper 100 entirely. This may also allow for an elongated member to be inserted into the gripper 100.

The through hole 12 is of a size and dimension which allows for the elongated member to be guided through the guide 1 and also allows for the elongated member to be inserted and/or removed from the guide 1. In some examples, there is only one hole in the guide 1, i.e. the elongated member cannot extend through the guide 1.

FIG. 3 shows a cut-away view of a schematic illustration of the gripper according to some example implementations as described herein.

It can be seen in FIG. 3 that the elongated member 6 extends though the guide 1 of the gripper via the through holes 12 mentioned above. This may allow for the elongated member 6 to be of a length substantially longer than the distance between the through holes 12 in the guide 1. This may be particularly advantageous in some scenarios. In particular, if the elongated member 6 is moved through the holes 12 via a “shuffling” technique where the elongated member 6 is fed through the holes 12 in gradual steps, the elongated member 6 being longer than the distance between the holes 12 may allow for the elongated member to be fed in gradual steps through the said holes 12 to achieve this technique.

It can be further seen in FIG. 3 that the assembly pins 4 are located within the first gripping component 2 and that these assembly pins 4 will allow for the first gripping component 2 to be coupled to the guide 1. In this example, the assembly pins 4 are pins but they may additionally or alternatively be a nut and bolt, a screw, a hinge, a bayonet coupling, a welding or any other suitable type of coupling or any combination thereof.

The elongated member 6 may be any elongated member which is preferably suitable for endovascular purposes, in particular a catheter or a guide wire. The elongated member 6 is not limited to the endovascular purposes and may alternatively be any type of elongated member which requires to be gripped.

Furthermore, in this example, there are two springs 5 coupled to the second gripping component 3. There may be a single spring 5 or any number of springs 5 which allow for the functioning of the gripper 100 as described in the present disclosure.

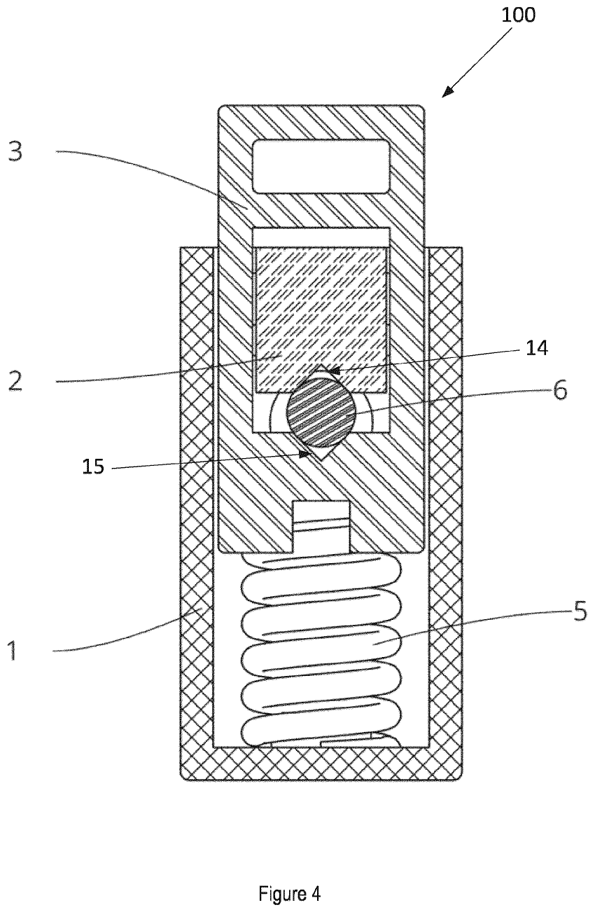

FIG. 4 shows a cross-sectional view of a schematic illustration of parts of the gripper according to some example implementations as described herein.

In this example, the first gripping component 2 and second gripping component 3 each comprises a respective recess 14, 15. The recesses are configured to accommodate at least a portion of the elongated member 6 within the gripper 100. In this example, both of the recesses 14, 15 are V-shaped but they may alternatively be any suitable shape such as, for example, cuboidal, semi-cylindrical or a bespoke shape.

In some examples, the first gripping component 2 comprises a metallic portion, wherein the metallic portion is configured to contact at least a portion of the elongated member 2. The metallic portion preferably comprises aluminum, and in particular 7075-t6 aluminum. The elongated member preferably comprises a plastic or polymer contactable with the metallic portion. This in turn may provide for a particularly strong gripping of the elongated member 6, thereby reducing the likelihood of the elongated member 6 moving while being gripped by the gripper 100.

In this example, the first gripping component 2 is partially housed by the second gripping component 3. This may allow for the movement of the second gripping component 3 to be limited as it may contact the first gripping component 2. This may result in a reduction of unwanted forces being exerted on the second gripping component 3, thereby extending the lifespan of the second gripping component 3.

The second gripping component 3 is configured to be slideable past both the guide 1 and the first gripping component 2. This may allow for the second gripping component 3 to be limited to moving in a single axis, i.e. the axis of the exertion of the biasing force, thereby reducing unwanted movement by the second gripping component 3 during movement of the second gripping component 3 and while the elongated member 6 is being gripped. This may also result in a reduction of unwanted forces being exerted on the elongated member 6, thereby also increasing the lifespan of the elongated member 6.

In FIG. 4, the gripper 100 is in the gripping position, i.e. the elongated member 6 is being gripped between the first 2 and second 3 gripping components. As described above, the spring 5 provides a biasing force which biases the second gripping component 3 towards the first gripping component 2, thereby keeping the elongated member 6 gripped. In some examples, a force exerted by a pneumatic cylinder and/or an electric motor and/or any other suitable component against the biasing force allows for the elongated member 6 to be moved, removed or inserted into the gripper. Once this force has been removed, the biasing force biases the second gripping component 3 and the elongated member 6 is once again gripped by the gripper 100. This also means that the elongated member 6 is gripped via a compressive force. The use of compressive forces may allow for a more secure gripping and/or securing of the elongated member 6 and may allow for the lifetime of the elongated member 6 to be increased, as it is not subjected to shear or tension forces.

The V-shaped recesses 14, 15 allow for the elongated member 6 to be contacted, along four contact lines, by said recesses 14, 15. The use of contact lines may allow for the compressive force to be exerted along the portion of the elongated member 6 which is contacted by the recesses 14, 15, thereby reducing the force that is exerted on any single portion of the elongated member 6. This may allow for the elongated member 6 to have a longer lifespan, as the compressive force is not focused at a single point. It may also allow for a more secure gripping of the elongated member 6, as the elongated member 6 is contacted along a portion of said elongated member 6 by the first 2 and second 3 gripping components, thereby increasing the area of contact where the elongated member 6 is contacted by the gripping components 2, 3.

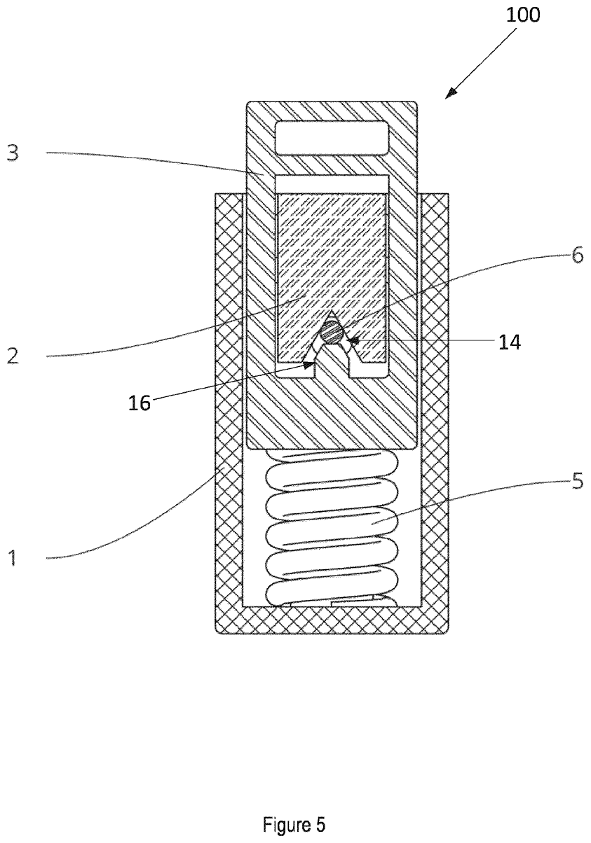

FIG. 5 shows a cross-sectional view of a schematic illustration of parts of the gripper according to some example implementations as described herein.

In this example, the first gripping component 2 comprises a recess 14, as described above, but the second gripping component alternatively comprises a protrusion 16. The protrusion may be formed via 3D printing but may additionally or alternatively be produced by milling and/or electrical discharge machining (EDM). In this example, the protrusion 16 comprises a truncated V-shape, wherein the truncated V-shape is configured to contact the elongated member 6. The sloped faces of the truncated V-shape are preferably angled at the same angle as the faces of the V-shaped recess 14. This may allow for a particularly secure gripping of the elongated member 6 when the elongated member 6 is being gripped. The protrusion may additionally (at one or more other portions) or alternatively comprise other shapes such as, for example, cuboidal, semi cylindrical, prismoidal or a bespoke shape.

As can be seen in FIGS. 4 and 5, a design of the gripper 100, and in particular the first 2 and second 3 gripping components may be altered depending on a parameter of the elongated member 6. The parameter may be, for example, a material of the elongated member 6, a dimension of the elongated member 6, a usage of the elongated member 6, a resilience of the elongated member 6 or any other suitable parameter (or, in case more parameters are taken into account, any combination thereof).

Furthermore, the biasing force provided by the spring 5 coupled to the second gripping component may be altered depending on one of these parameters. In a nonlimiting example, if the elongated member 6 is a guide wire, a biasing force of up to 5 Newton may be exerted by the spring 5 on the second gripping component and if the elongated member 6 is a catheter, the biasing force may be of up to 15 Newton. The biasing force may be altered via a replacement of the gripper 100 with a second gripper, wherein the springs 5 of the second gripper provide a biasing force which is suitable for the elongated member 6. Additionally or alternatively, the gripper 100 may further comprise a force sensor configured to sense a compressive force being exerted on the elongated member 6. The force sensor may then indicate, via audio, visual or haptic means to the user that the compressive force is too large for the elongated member 6. In some examples, the force sensor may automatically ungrip (release) the elongated member 6, i.e. provide a force against the biasing force and/or stop the gripper 100 from exerting more force onto the elongated member 6 by preventing a movement of the second gripping component 3. A user may be able to indicate to the force sensor compressive force limit via an input display on the gripper 100 and/or via a dial on the gripper 100 and/or via any other suitable method. In some examples, the user may input a type of elongated member 6, i.e. a catheter or a guide wire, and the force sensor may then automatically determine a compressive force limit based on a predetermined input to the force sensor.

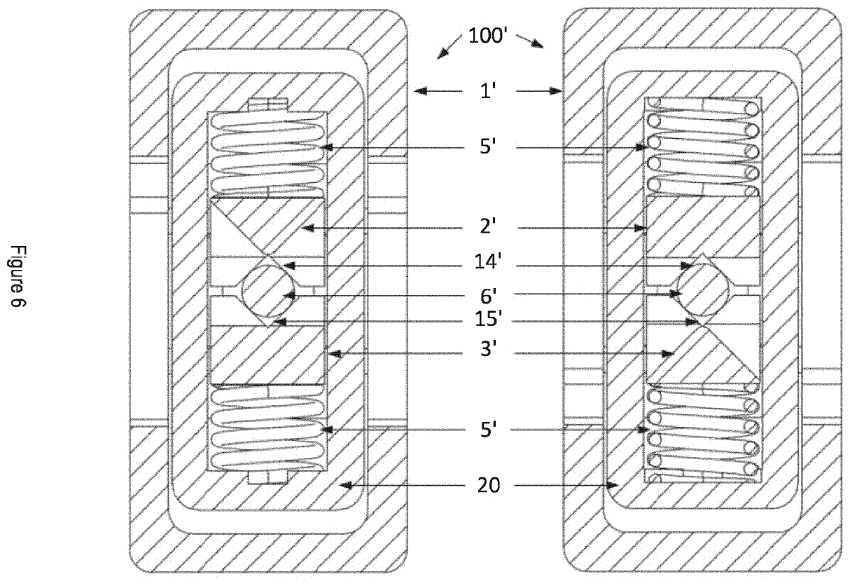

FIG. 6 shows cross-sectional views of a schematic illustration of parts of the gripper according to some example implementations as described herein.

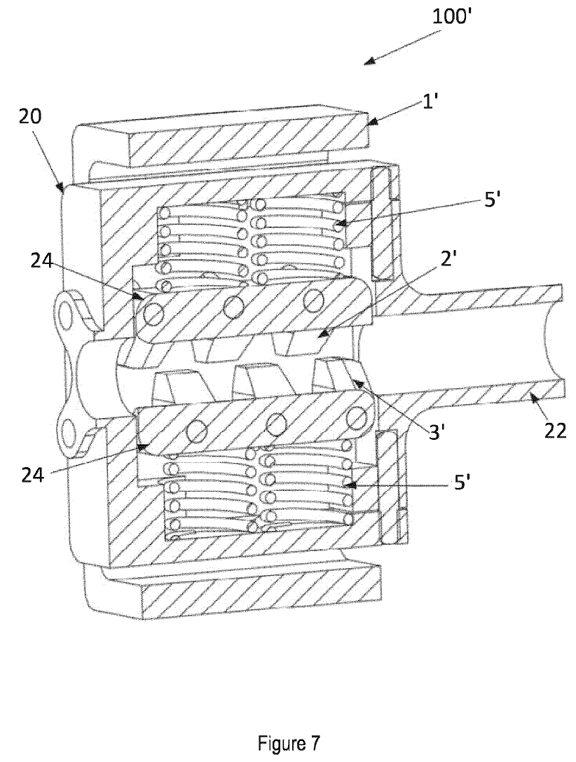

In FIGS. 6 and 7, features which are substantially similar to the features of the gripper of FIGS. 1 to 5 are indicated by the same reference numeral, but with a “ ” after it. For example, the spring of FIGS. 6 and 7 is marked with 5′. Any characteristics of these features mentioned in relation to FIGS. 6 and 7 can also apply to the corresponding features of FIGS. 1 to 5, and vice versa.

In the gripper 100′ of FIG. 6, there is a guide 1′ configured to guide not only the first and second gipping components 2′, 3′, but also an internal guide 20. This is described in more detail below. In the embodiments of FIGS. 6 and 7, both the first and second gripping components 2′, 3′ are coupled to springs 5′ and therefore, both the first and second gripping components 2′, 3′ are moveable in the same manner as the second gripping component 3 of FIGS. 1 to 5. As both the first and second gripping components 2′, 3′ are moveable, the inner guide 20 is configured to guide at least a portion of the first gripping component 2′ and at least a portion of the second gripping component 3′. The inner guide 20 may also be configured to guide a least a portion of at least one of the springs 5′. This may allow for parts of the gripper 100′ to be guided in a manner which reduces unwanted stress and shear forces during operation of the gripper 100′, thereby extending the lifetime of the gripper 100′. In some examples, the inner guide 20 guides only one, or some, of the first gripping component 2′, the second gripping component 3′ and at least one of the plurality of springs 5′. The first and second gripping components 2′, 3′ may be at least partially located within the inner guide 20, the inner guide 20 may be located within the guide 1′, and the inner guide 20 may be moveable with respect to the guide 1′.

The inner guide 20 may be moveable with respect to the guide 1′ in order to allow for fewer stress and shear forces to be experienced by the elongated member 6′. In this case, the combination of the inner guide 20 and the guide 1′ may act as a form of suspension and cushioning for the elongated member 6′ gripped by the gripper 100′. Alternatively, the inner guide 20 may be fixedly coupled to the guide 1′ via pins 4 such as those described above. Additionally or alternatively, the first or second gripping component 2′, 3′ may be stationary, or substantially stationary, with respect to the guide 1′. This may be achieved via said pins 4.

Additionally, the first and second gripping components 2′, 3′ have respective recesses 14′, 15′ that allow for the elongated member 6′ within the gripper 100′ to be gripped, similar to the recesses 14, 15 described above. In some examples, there may be a protrusion 16 instead of a recess 14, 15, as described above.

Similar to the gripper 100 of FIGS. 1 to 5, the gripper 100′ of FIGS. 6 and 7 is configured to grip the elongated member 6′ via at least a portion of the first gripping component 2′ and a portion of the second gripping component 3′ when at least one of the springs 5′ coupled to the first or second gripping component 2′, 3′ is actuated on by the actuating component (not shown in these figures).

The first and second gripping components 2′, 3′ coupled to their respective springs 5′ may have substantially cuboidal or prismatic designs. That is to say, the first gripping component may have a substantially prismatic design, whereas the second gripping component may have a substantially cuboidal design, and vice versa. However, any suitable design in any suitable combination may be used for the first and second gripping components 2′, 3′. The recesses 14′, 15′ may be incorporated into such designs. In some examples, the first and second gripping components 2′, 3′ may be of the cuboidal or prismatic design, and be coupled to a secondary component that comprises the recess 14′, 15′.

FIG. 7 shows a cut-away view of a schematic illustration of the gripper according to some example implementations as described herein.

In the gripper 100′ of FIG. 7, most components are substantially the same when compared to the gripper of FIG. 6. The gripper 100′ of FIG. 7 further comprises a guide tube 22. This guide tube 22 may be of a suitable dimension to accept the elongated member 6′. The elongated member 6′ can then be guided towards the center of the gripper 100′ comprising, for example, the first and second gripping components 2′, 3′.

In the example of FIG. 7, the first and second gripping components 2′, 3′ are each coupled to a corresponding, respective block 24, which in turn is coupled to a plurality of springs 5′. In some examples, there may only be one spring 5′ coupled to at least one of the blocks 24.

The first and second gripping components 2′, 3′ comprise a truncated V-shape, similar to the protrusion 16 described above, but may have any suitable design. Indeed, at least one of the first and/or second gripping components 2′, 3′ may comprise a recess 14′, 15′ as described above. However, the first and second gripping components 2′, 3′ are offset from each other so that they do not directly contact each other. That is to say, when the first and second gripping components 2′, 3′ are moved towards each other, via extension of at least one of the springs 5′, the first and second gripping components 2′, 3′ slot between each other similar to teeth of two gears, or to a zip. This may allow for the elongated member 6′ within the gripper 100′ to be gripped in a particularly strong manner. That is to say, in this configuration, when gripped, the elongated member is crimped between the first and second gripping components 2′, 3′. Alternatively, the elongated member 6′ may be gripped in such a way that, when viewed from the side, the elongated member 6′ has the form of a sine wave or generally a wave-like form. This may allow for the elongated member 6′ to be gripped in a particularly strong and stable manner. This may also allow for more contact points, thereby further securing the elongated member 6′ within the gripper 100′. Alternatively, the first and second gripping components 2′, 3′ may contact each other.

The force of the grip may be influenced by the strength of the springs 5′ and/or the distance that the springs 5′ are allowed to be extended when acted upon by the actuating component. That is to say, if the springs were extended fully, and, when viewed from the side, the elongated member 6′ has the form similar to a square wave, the elongated member 6′ may be very secure. However, this could lead to damage to the elongated member 6′. Therefore, the extension of the springs 5′ may be altered based on, for example, the material, fragility and diameter of the elongated member 6′ to be gripped.

Additionally, in FIG. 7, there are six gripping components 2′, 3′ with three gripping components 2′ being coupled to one of the blocks 24 and the other three gripping components 3′ being coupled to the other block 24. However, it will be realized by the skilled person that there may be any number of gripping components 2′, 3′ within the gripper 100′ and/or that the number of gripping components 2′, 3′ coupled to each respective block 24 may not be equal. This is to say, in a non-limiting example, there may be two gripping components 2′ coupled to one of the blocks 24 and four gripping components 3′ being coupled to the other block 24.

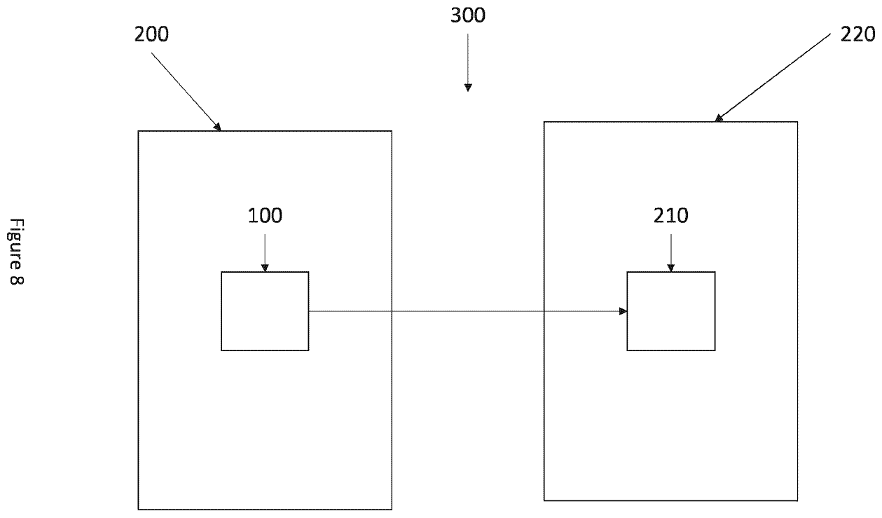

FIG. 8 shows a schematic block diagram of an endovascular system according to some example implementations as described herein.

The endovascular system 300 comprises a first 200 and a second 220 endovascular instrument. The first 200 and second 220 endovascular instruments may be located in discrete locations and a movement of the first endovascular instrument 200 may be translated (mimicked) to a corresponding movement in the second endovascular instrument 220. In particular, a movement of the gripper 100 in the first endovascular instrument may be translated into a movement in an instrument 210 in the second endovascular instrument 220. In some examples, this instrument 210 is a second gripper.

No doubt many other effective alternatives will occur to the skilled person. It will be understood that the invention is not limited to the described embodiments and encompasses modifications apparent to those skilled in the art and lying within the scope of the claims appended hereto.

1. A gripper for gripping an elongated member of an endovascular system, wherein the gripper comprises:
a first gripping component configured to contact at least a first portion of a said elongated member on a first side of said elongated member;
a second gripping component configured to contact at least the first portion of said elongated member on a second side of said elongated member, wherein the first side is different from the second side;
a guide configured to guide, during a movement of the second gripping component, at least a first portion of the second gripping component; and
an actuating component coupled to the second gripping component, wherein the second gripping component is moveable between a first position and a second position based on an actuating force provided to the second gripping component via the actuating component, and wherein at least the first portion of the second gripping component is guidable by the guide during movement of the second gripping component between the first position and the second position;
wherein the first gripping component is configured to stay stationary or substantially stationary with respect to the guide,
wherein the second gripping component comprises a first surface opposite to a first surface of the first gripping component,
wherein, when the second gripping component is in the first position, said elongated member is grippable between the first surface of the first gripping component and the first surface of the second gripping component, and
wherein, when the second gripping component is in the second position, said elongated member is not grippable between the first surface of the first gripping component and the first surface of the second gripping component.

2. The gripper of claim 1, wherein the first surface of the first gripping component comprises a first recess configured to accommodate at least the first portion of said elongated member on the first side of said elongated member, in particular wherein the first recess is V-shaped.

3. (canceled)

4. The gripper of claim 1, wherein the first surface of the second gripping component comprises a second recess configured to accommodate at least the first portion of said elongated member on the second side of said elongated member, in particular wherein the second recess is V-shaped.

5. (canceled)

6. The gripper of claim 4, wherein the first surface of the first gripping component comprises a first recess configured to accommodate at least the first portion of said elongated member on the first side of said elongated member, wherein at least the first portion of said elongated member is contactable, via the first surface of the first gripping component on the first side of said elongated member and the first surface of the second gripping component on the second side of said elongated member, at four contact lines via the first and second recesses, in particular wherein the four contact lines are each separated by 90°, and wherein the four contact lines are parallel to one another.

7. (canceled)

8. The gripper of claim 4, wherein the first surface of the first gripping component comprises a first recess configured to accommodate-at least the first portion of said elongated member on the first side of said elongated member, wherein at least the first portion of said elongated member is contactable, via the first surface of the first gripping component on the first side of said elongated member and the first surface of the second gripping component on the second side of said elongated member, at four contact points via the first and second recesses.

9. The gripper of claim 8, wherein the four contact points are each separated by 90°, and wherein the four contact points span a quadrilateral around a central longitudinal axis of said elongated member.

10. The gripper of claim 1, wherein the first surface of the second gripping component comprises a protrusion configured to contact at least the first portion of said elongated member on the second side of said elongated member, in particular wherein the protrusion comprises a truncated V shape.

11. (canceled)

12. The gripper of claim 10, wherein the first surface of the first gripping component comprises a first recess configured to accommodate at least the first portion of said elongated member on the first side of said elongated member, wherein at least the first portion of said elongated member is contactable, via the first surface of the first gripping component on the first side of said elongated member and the first surface of the second gripping component on the second side of said elongated member, at two contact lines via the first recess and one contact line via the protrusion.

13. The gripper of claim 12, wherein the three contact lines are each separated by 120°, and wherein the three contact lines are parallel to one another.

14. The gripper of claim 10, wherein the first surface of the first gripping component comprises a first recess configured to accommodate at least the first portion of said elongated member on the first side of said elongated member, wherein at least the first portion of said elongated member is contactable, via the first surface of the first gripping component on the first side of said elongated member and the first surface of the second gripping component on the second side of said elongated member, at two contact points via the first recess and one contact point via the protrusion, in particular wherein the three contact points are each separated by 120°, and wherein the three contact points span a triangle around a central longitudinal axis of said elongated member.

15. (canceled)

16. The gripper of claim 1, wherein the actuating component comprises a resilient member, in particular a spring;
and/or wherein the guide comprises a through hole configured to receive said elongated member between the first surface of the first gripping component and the first surface of the second gripping component;
and/or wherein at least the first portion of said elongated member is grippable between the first surface of the first gripping component and the first surface of the second gripping component;
and/or wherein the second gripping component and/or the guide comprises a photopolymer and/or comprises a 3D-printed portion;
and/or wherein the first gripping component comprises a metallic portion, wherein at least a portion of the metallic portion is configured to contact at least the first portion of said elongated member;
and/or wherein upon said movement of the second gripping component, based on the actuating force provided to the second gripping component via the actuating component, the first surface of the second gripping component is moveable towards the first surface of the first gripping component.

17. (canceled)

18. (canceled)

19. (canceled)

20. (canceled)

21. (canceled)

22. The gripper of claim 1, further comprising an assembly pin configured to couple
(i) the first gripping component and the guide to each other, and/or
(ii) the first gripping component and a component external to the gripper to each other, and/or
(iii) the guide and the component external to the gripper to each other.

23. The gripper of claim 1, wherein the guide comprises a first portion and a second portion, wherein the first portion is configured to guide at least the first portion of the second gripping component and to guide at least the first portion of said elongated member, wherein the second portion of the guide comprises a section configured to guide at least a second portion of said elongated member, and wherein the first and second portions of said elongated member are distinct portions;
and/or wherein the gripper is situated in a disposable cassette;
and/or wherein the actuating force provided to the second gripping component via the actuating component is dependent on at least one parameter of said elongated member;
and/or wherein said elongated member is a guide wire and/or a catheter;
and/or wherein the first gripping component and/or the second gripping component and/or the guide are at least partially coated in a polymer.

24. (canceled)

25. (canceled)

26. (canceled)

27. (canceled)

28. The gripper of claim 1, further comprising an assembly pin configured to couple
(i) the first gripping component and the guide to each other, and/or
(ii) the first gripping component and a component external to the gripper to each other, and/or
(iii) the guide and the component external to the gripper to each other, and wherein the gripper further comprises a sensor configured to indicate to the external component an actuation of the actuating component.

29. The gripper of claim 1, wherein the first gripping component is at least partially housed by the second gripping component;
and/or wherein the guide is further configured to guide at least a portion of the actuating component and/or at least a first portion of the first gripping component;
and/or wherein the second gripping component is slideable past the first gripping component and the guide during the movement of the second gripping component between the first position and the second position and/or between the second position and the first position;
and/or wherein the movement of the second gripping component towards the second position is limitable upon a contact between the first gripping component and the second gripping component;
and/or wherein the movement of the second gripping component towards the first position is limitable upon a contact between the second gripping component and said elongated member.

30. (canceled)

31. (canceled)

32. (canceled)

33. (canceled)

34. The gripper of claim 1, wherein the first surface of the first gripping component comprises a first recess configured to accommodate at least the first portion of said elongated member on the first side of said elongated member, wherein a movement of said elongated member is limitable by the first recess and/or the second recess when the second gripping component is in the second position.

35. The gripper of claim 1, wherein the first gripping component comprises a metallic portion, wherein at least a portion of the metallic portion is configured to contact at least the first portion of said elongated member, wherein the metallic portion comprises 7075-t6 aluminum.

36. The gripper of claim 1, wherein the first and second gripping components are at least partially located within an inner guide, wherein the inner guide is located within the guide, and wherein the inner guide is moveable with respect to the guide, in particular wherein the first and second gripping components are offset from each other along a longitudinal axis of the elongated member, and when the second gripping component moves between the first position and the second position, the second gripping component does not contact the first gripping component, more particularly wherein the first and second gripping components are arranged in a zipper configuration.

37. (canceled)

38. (canceled)

39. The gripper of claim 1, wherein at least a portion of the first and/or second gripping component contactable with the elongated member comprises a coating layer, in particular wherein the coating layer comprises one or more of:
a rubber;
a textile;
a fabric;
a polymer layer;
a non-polymer layer;
a paint layer;
and a composite.

40. (canceled)

41. An endovascular system comprising: a first endovascular instrument; and
a second endovascular instrument;
wherein the first and/or second endovascular instrument comprises a gripper for gripping an elongated member of an endovascular system, wherein the gripper comprises:
a first gripping component configured to contact at least a first portion of a said elongated member on a first side of said elongated member;
a second gripping component configured to contact at least the first portion of said elongated member on a second side of said elongated member, wherein the first side is different from the second side;
a guide configured to guide, during a movement of the second gripping component, at least a first portion of the second gripping component; and
an actuating component coupled to the second gripping component, wherein the second gripping component is moveable between a first position and a second position based on an actuating force provided to the second gripping component via the actuating component, and wherein at least the first portion of the second gripping component is guidable by the guide during movement of the second gripping component between the first position and the second position;
wherein the first gripping component is configured to stay stationary or substantially stationary with respect to the guide,
wherein the second gripping component comprises a first surface opposite to a first surface of the first gripping component,
wherein, when the second gripping component is in the first position, said elongated member is grippable between the first surface of the first gripping component and the first surface of the second gripping component, and
wherein, when the second gripping component is in the second position, said elongated member is not grippable between the first surface of the first gripping component and the first surface of the second gripping component,
in particular wherein the system further comprises the elongated member, wherein the elongated member is a guide wire and/or a catheter, and/or wherein the first endovascular instrument is a first robotic endovascular instrument and/or the second endovascular instrument is a second robotic endovascular instrument.

42. (canceled)

43. (canceled)
